(19) United States
(12) Patent Application Publication
(10) Pub. No.: US 2024/0094191 A1
(43) Pub. Date: Mar. 21, 2024

(54) DIETARY HABIT EVALUATION SYSTEM AND DIETARY HABIT EVALUATION METHOD

(71) Applicant: INSTITUTE FOR HEALTH RESTORATION CO., LTD., Kyoto (JP)

(72) Inventors: Yukio YAMORI, Hyogo (JP); Mari MORI, Hyogo (JP)

(73) Assignee: INSTITUTE FOR HEALTH RESTORATION CO., LTD., Kyoto (JP)

(21) Appl. No.: 18/263,977

(22) PCT Filed: Jan. 28, 2022
(86) PCT No.: PCT/JP2022/003242

§ 371 Date: Aug. 2, 2023

(30) Foreign Application Priority Data
Feb. 3, 2021 (JP) 2021-015501

(51) Int. Cl. G01N 33/493

(52) U.S. Cl. CPC G01N 33/493

(57) ABSTRACT

An acquisition control unit acquires spot urine nutritional component indexes of nutritional component indexes contained in one spot urine excreted by a human. The nutritional component indexes two or more selected from a sodium/potassium ratio indicating a ratio, a magnesium/creatinine ratio, an urea-nitrogen/creatinine ratio, and a sodium/creatinine ratio. The determination control unit compares the spot urine nutritional component index with a reference value preset for the spot urine nutritional component index for each of spot urine nutritional component indexes, and determines whether or not the spot urine nutritional component index is equal to or more than the reference value. The decision control unit decides a diet evaluation pattern indicating a balance of the diet of the human from the determination result for each of the determined spot urine nutritional component indexes. The output control unit outputs a diet evaluation comment improving the balance of the diet of the human.

### TECHNICAL FIELD

The present invention relates to a dietary habit evaluation system and a dietary habit evaluation method.

### BACKGROUND ART

Conventionally, there are various techniques for measuring a specific component concentration contained in human urine and for evaluating a dietary habit of the human. For example, JP-A-2006-29819 (PTL1) discloses a urine analysis method for calculating a specific component excretion amount that a human excretes per day. First, this urine analysis method measures an amount of the first urine excreted by a human after waking up and a concentration of the specific component, and calculates a specific component excretion amount in the first urine after waking up. Next, this urine analysis method obtains an elapsed time from urination before bedtime to the first urination after waking up, and converts a specific component excretion amount to a regulation time equivalent based on a ratio of the elapsed time to a predetermined regulation time. Then, the urine analysis method calculates a specific component excretion amount that a human excretes per day based on the converted regulation time equivalent. For example, the specific component is one or more of sodium, potassium and calcium. In this way, by biometric information (the specific component excretion amount) measured without disrupting daily lifestyle habit, it is possible to get specific lifestyle improvement information that is necessary and concrete for the prevention and the management of the lifestyle-related disease even if it is a commoner without the medical knowledge.

Further, JP-A-2006-126184 (PTL2) discloses a component intake measuring device comprising a data acquisition unit, a specific component excretion equivalent calculation unit, a specific component intake calculation unit, and an output unit. The data acquisition unit acquires urine data including a sleep elapsed time which is an elapsed time from a time when it was urinated in the last before a predetermined bedtime period and a time when it was urinated in the first after the predetermined bedtime period, an amount of excreted urine during the sleep elapsed time, and a specific component concentration of the urine. The specific component excretion equivalent calculation unit calculates a specific component excretion equivalent which is an amount of the specific component excreted by a human in a regulation time, from the acquired amount of excreted urine, the specific component concentration, and predetermined regulation time. The specific component intake calculation unit estimates a daily specific component intake that a human takes in a day from the calculated specific component excretion equivalent. The output unit outputs a measurement result including the daily specific component intake. In this way, by measuring the specific component in urine excreted in daily life, it is possible to obtain lifestyle improvement information necessary for the prevention and management of lifestyle-related diseases.

Further, WO 13/021695 (PTL3) discloses a urine component analyzer comprising a correlation storage unit, a data input unit, and a calculation unit. The correlation storage unit stores data representing a correlation from a concentration ratio of a first specific component to a second specific component in one urine excreted by a human to a concentration ratio of a first specific component and a second specific component in total urine excreted in a day when all urine excreted in a day is collected. The data input unit inputs data representing a concentration ratio of the first specific component to the second specific component in one urine excreted by the subject. The calculation unit calculates a concentration ratio of the first specific component to the second specific component in total urine excreted in a day when all urine excreted in a day by the subject is collected with the correlation stored by the correlation storage unit based on the concentration ratio of the first specific component to the second specific component in one urine of the subject obtained through the data input unit. In this way, it is possible to easily obtain the concentration ratio between two specific components in the all urine excreted by the subject in a day.

Further, JP-A 2013-36817 (PTL4) discloses a urine component analyzer comprising a correlation storage unit, a total urine amount acquisition unit, a data input unit, a first calculation unit, and a second calculation unit. The correlation storage unit stores data representing a concentration ratio of a concentration of a specific component in one urine excreted by a human to a concentration of specific component in all urine excreted in one day when all urine excreted by the human in one day is collected. The total urine amount acquisition unit acquires a total urine amount excreted by a subject in one day based on conversion or a database. The data input unit inputs data representing a concentration of the specific component in one urine excreted by the subject. The first calculation unit converts and obtains a concentration of the specific component in the total urine in a day when all urine excreted by the subject in one day is collected with the correlation stored by the correlation storage unit based on the concentration of the specific component in one urine of the subject obtained through the data input unit. The second calculation unit multiplies the total urine amount acquired by the total urine amount acquisition unit by the concentration of the specific component in the total urine in a day obtained by the first calculation unit, and calculates an amount of excretion of the specific component in the total urine of the subject in a day. In this way, it is possible to simply and accurately obtain the amount of excretion of the specific component in the total urine of the subject in a day.

Also, NPL 1 (Yukio Yamori et al, “DIETARY RISK FACTORS OF STROKE AND HYPERTENSION IN JAPAN—Part 1: Methodological Assessment of Urinalysis for Dietary Salt and Protein Intakes—”, Japanese Circulation Journal, Vol. 46, September 1928, p 933-938), Non-Patent Literature 2 (Yukio Yamori et al, “DIETARY RISK FACTORS OF STROKE AND HYPERTENSION IN JAPAN—Part 2: Validity of Urinalysis for Dietary Salt and Protein Intakes under a Field Condition—”, Japanese Circulation Journal, Vol. 46, September 1928, p. 939-943), Non-Patent Literature 3 (Yukio Yamori et al, “DIETARY RISK FACTORS OF STROKE AND HYPERTENSION IN JAPAN—Part 3: Comparative Study on Risk Factors between Farming and Fishing Villages in Japan—”, Japanese Circulation Journal, Vol. 46, September 1928, p 944-948) disclose to evaluate dietary habit by using a value obtained by dividing a concentration of a specific component in 24-hour urine by a concentration of creatinine in urine.

Also, NPL 4 (Yukio Yamori et al, “An inverse association between magnesium in 24-hour urine and cardiovascular risk factors in middle-aged subjects in 50 CARDIAC Study populations”, Hypertension Research (2015) 38, p. 219-225) discloses that a risk of a cardiovascular disease increases if magnesium/creatinine ratio in 24-hour urine is high.

Also, NPL 5 (Yukio Yamori et al. “An inverse association between magnesium in 24-hour urine and cardiovascular risk factors in middle-aged subjects in 50 CARDIAC Study populations”, Hypertens Res. 2015 March; 38(3): p 219-225) discloses that the higher Mg/Cre ratio which is the ratio of magnesium concentration to creatinine concentration in 24-hour urine, the lower a risk factor such as the body weight index (BMI), blood pressure (BP), serum total cholesterol (TC), obesity, hypertension and hypercholesterolemia reduce.

Also, Non-Patent Literature 6 (Okuda M et al. “Estimation of daily sodium and potassium excretion from overnight urine of Japanese children and adolescents”, Environ Health Prev Med. 2020 Nov. 27; 25(1): p. 74) discloses that it is possible to estimate sodium and potassium excretion amount in a day by making sodium concentration, potassium concentration, creatinine concentration, height, weight and age, variables.

### CITATION LIST

### Patent Literature

[PTL1] JP-A-2006-29819
[PTL2] JP-A-2006-126184
[PTL3] International Publication No. 13/021695
[PTL4] JP-A-2013-36817

### Non Patent Literature

[NPL1] Yukio Yamori et al, “DIETARY RISK FACTORS OF STROKE AND HYPERTENSION IN JAPAN—Part 1: Methodological Assessment of Urinalysis for Dietary Salt and Protein Intakes—”, Japanese Circulation Journal, Vol. 46, September 1928, p. 933-938
[NPL2] Yukio Yamori et al, “DIETARY RISK FACTORS OF STROKE AND HYPERTENSION IN JAPAN—Part 2: Validity of Urinalysis for Dietary Salt and Protein Intakes under a Field Condition—”, Japanese Circulation Journal, Vol. 46, September 1928, p. 939-943
[NPL3] Yukio Yamori et al, “DIETARY RISK FACTORS OF STROKE AND HYPERTENSION IN JAPAN—Part 3: Comparative Study on Risk Factors between Farming and Fishing Villages in Japan—”, Japanese Circulation Journal, Vol. 46, September 1928, p. 944-948
[NPL4] Yukio Yamori et al, “An inverse association between magnesium in 24-hour urine and cardiovascular risk factors in middle-aged subjects in 50 CARDIAC Study populations”, Hypertension Research (2015) 38, 219-225
[NPL5] Yukio Yamori et al. “An inverse association between magnesium in 24-hour urine and cardiovascular risk factors in middle-aged subjects in 50 CARDIAC Study populations”, Hypertens Res. 2015 March; 38(3):219-225
[NPL6] Okuda M et al. “Estimation of daily sodium and potassium excretion from overnight urine of Japanese children and adolescents”, Environ Health Prev Med. 2020 Nov. 27; 25(1): p. 74

### SUMMARY OF INVENTION

### Technical Problem

For example, by taking fasting blood samples and questionnaires on dietary (eating) habit, etc. human nutritional surveys are conducted. But the surveys have the problems that there are troublesome and time-consuming, and it is difficult to appropriately analyze the types of the diet that human take by mouth.

The technique described in PL 1 uses the first urine after waking up, but there is a problem that human tends to forget to take the first urine after waking up. The technique described in PL 2 uses the sleep elapsed time and the urine in the sleep elapsed time, but similarly there is a problem that human tends to forget to take urine.

The technique described in PL 3 converts the concentration ratio of the first specific component to the second specific component in one urine of the subject to the concentration ratio of the first specific component to the second specific component in total urine excreted in a day. The difference between the converted concentration ratio of the first specific component to the second specific component in total urine excreted in a day and an actual concentration ratio of the first specific component to the second specific component in total urine excreted in a day is large, there is a problem that it is impossible to convert with high accuracy. The technique described in PL 4 converts the concentration of the specific component in one urine of the subject in a day to the concentration of the specific component in the total urine in a day. But similarly, there is a problem that the difference between the converted concentration of the specific components in one urine of the subject in a day and an actual concentration of the specific component in total urine in a day is large.

The techniques described in NPLs 1-4 evaluate dietary habit by the concentration of specific component/creatinine ratio in 24-hour urine, and evaluate the risk of cardiovascular disease. There is a problem that 24-hour urine is required. The techniques described in NPLs 5 and 6 also have the problem of requiring 24-hour urine.

In order to accurately evaluate human dietary habit, it is preferable to collect the 24-hour urine of the human in a day, and to calculate the specific component concentration and the specific component excretion amount in 24-hour urine. But, if human forgets to collect urine even once, the urine will not be 24-hour urine, there is a problem that 24-hour urine collection itself is difficult.

The conventional human dietary habit evaluation by using spot urine has the problem of low accuracy and lack of reliability regarding the relationship with the risk of lifestyle-related diseases. There is a problem that there are few types of nutritional components in spot urine, and the nutritional components that can be evaluated are limited, and it is not possible to evaluate the human diet as a whole.

The present invention was created as a solution for the problems and aims at providing a dietary habit evaluation system and a dietary habit evaluation method that can easily and entirely evaluate human dietary habit by using a human spot urine.

### Solution to Problem

A dietary habit evaluation system according to the present invention includes an acquisition control unit, a determination control unit, a decision control unit, and an output control unit. The acquisition control unit acquires spot urine nutritional component indexes of nutritional component indexes contained in one spot urine excreted by a human, the nutritional component indexes two or more selected from a sodium/potassium ratio indicating a ratio of a sodium ion concentration and a potassium ion concentration, a magnesium/creatinine ratio indicating a ratio of a magnesium ion concentration to a creatinine concentration, an urea-nitrogen/creatinine ratio indicating a ratio of a urea nitrogen concentration to the creatinine concentration, and a sodium/creatinine ratio indicating a ratio of the sodium ion concentration to the creatinine concentration. The determination control unit compares the spot urine nutritional component index with a reference value preset for the spot urine nutritional component index for each of spot urine nutritional component indexes, and determines whether or not the spot urine nutritional component index is equal to or more than the reference value for each of spot urine nutritional component indexes. The decision control unit decides a diet evaluation pattern indicating a balance of the diet of the human from the determination result for each of the determined spot urine nutritional component indexes. The output control unit outputs a diet evaluation comment improving the balance of the diet of the human for the diet evaluation pattern.

The dietary habit evaluation method according to the present invention includes an acquisition control step, a determination control step, a decision control step, and an output control step. Each of the steps in the dietary habit evaluation method according to the present invention corresponds to each of the control units in the dietary habit evaluation system according to the present invention.

### Advantageous Effects of Invention

According to this invention, it is possible to easily and entirely evaluate human dietary habit by using a human spot urine.

### BRIEF DESCRIPTION OF DRAWINGS

FIG. 1 is a table showing a relationship between 24-hour urine nutritional component index, spot urine nutritional component index, sex of a subject, the number of subjects and a correlation coefficient between the 24-hour urine nutritional component index and the spot urine nutritional component index.

FIG. 2 is a graph showing a relationship between sodium/potassium ratios of spot urine and sodium/potassium ratios of 24-hour urine when the subjects are male and female.

FIG. 3 is a graph showing a relationship between the sodium/potassium ratios of the spot urine and the sodium/potassium ratios of the 24-hour urine when the subjects are male only.

FIG. 4 is a graph showing a relationship between the sodium/potassium ratios of the spot urine and the sodium/potassium ratios of the 24-hour urine when the subjects are female only.

FIG. 5 is a graph showing a relationship between magnesium/creatinine ratios of spot urine and magnesium/creatinine ratios of 24-hour urine when the subjects are male and female.

FIG. 6 is a graph showing a relationship between the magnesium/creatinine ratios of the spot urine and the magnesium/creatinine ratios of the 24-hour urine when the subjects are male only.

FIG. 7 is a graph showing a relationship between the magnesium/creatinine ratios of the spot urine and the magnesium/creatinine ratios of the 24-hour urine when the subjects are female only.

FIG. 8 is a graph showing a relationship between urea-nitrogen/creatinine ratios of spot urine and urea-nitrogen/creatinine ratios of 24-hour urine when the subjects are male and female.

FIG. 9 is a graph showing a relationship between urea-nitrogen/creatinine ratios of the spot urine and urea-nitrogen/creatinine ratios of the 24-hour urine when the subjects are male only.

FIG. 10 is a graph showing a relationship between urea-nitrogen/creatinine ratios of the spot urine and urea-nitrogen/creatinine ratios of the 24-hour urine when the subjects are female only.

FIG. 11 is a graph showing a relationship between sodium/creatinine ratios of spot urine and sodium/creatinine ratios of 24-hour urine when the subjects are male and female.

FIG. 12 is a graph showing a relationship between sodium/creatinine ratios of the spot urine and sodium/creatinine ratios of the 24-hour urine when the subjects are male only.

FIG. 13 is a graph showing a relationship between sodium/creatinine ratios of the spot urine and sodium/creatinine ratios of the 24-hour urine when the subjects are female only.

FIG. 14 is a conceptual diagram and a function block diagram of the dietary habit evaluation system according to an embodiment of the present invention.

FIG. 15 is a flowchart of the dietary habit evaluation method according to an embodiment of the present invention.

FIG. 16 shows a pattern sheet according to an embodiment of the present invention.

FIG. 17 shows a case when numerical values are reflected in the pattern sheet according to an embodiment of the present invention.

FIG. 18 shows a comment sheet according to the embodiment of the present invention.

FIG. 19 shows a BMI sheet according to the embodiment of the invention.

FIG. 20 shows a good habit questionnaire sheet according to the embodiment of the invention.

FIG. 21 shows a bad habit questionnaire sheet according to the embodiment of the invention.

FIG. 22 is a diagram showing a sheet of reference diet evaluation patterns and comments when dealing with two types of spot urine nutritional component indexes.

FIG. 23 is a diagram showing a sheet of reference diet evaluation patterns and comments when dealing with three types of spot urine nutritional component indexes.

FIG. 24 is a diagram showing other sheet of reference diet evaluation patterns and comments when dealing with three types of spot urine nutritional component indexes.

### DESCRIPTION OF EMBODIMENTS

The preferred embodiments of the present invention will be explained below according to the attached drawings; thereby the present invention will be clearly understood. The embodiments below are examples materializing the present invention, and do not limit the technical scope of the present invention.

The present inventors have been grasping a nutritional status of a human by a level of nutritional component concentration in 24-hour urine by evaluating the nutritional component concentration in 24-hour urine and a dietary habit of the human who excreted the urine, and finding for many years that the risk of lifestyle-related diseases such as myocardial infarction, hypertension, circulatory disease, and stroke. On the other hand, there is a problem that it is difficult to take 24-hour urine itself.

Therefore, the present inventors searched for an index to replace the nutritional component concentration in 24-hour urine, and discovered that the one spot urine nutritional component index, which will be described later, correlates with the index of nutrients in 24-hour urine.

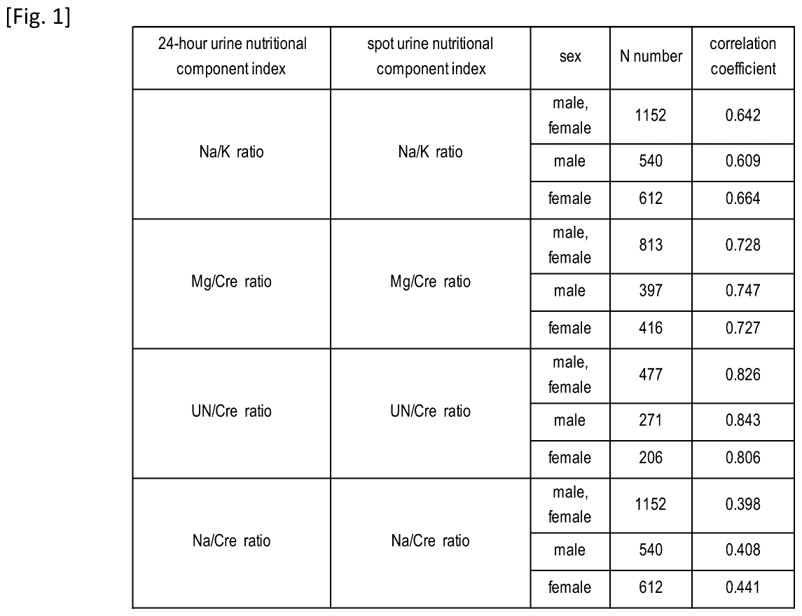

FIG. 1 is a table showing a relationship between 24-hour urine nutritional component indexes, spot urine nutritional component indexes, sex of a subject, the number (N number) of subjects and a correlation coefficient between the 24-hour urine nutritional component index and the spot urine nutritional component indexes. Here, as nutritional component indexes, a sodium/potassium ratio (Na/K ratio) indicates a ratio of (dividing) a sodium ion concentration in urine to (by) a potassium ion concentration in urine, a magnesium/creatinine ratio (Mg/Cre ratio) indicates a ratio of a magnesium ion concentration in urine to a creatinine concentration in urine, an urea-nitrogen/creatinine ratio (UN/Cre ratio) indicates a ratio of a urea-nitrogen (UN) concentration in urine to a creatinine concentration in urine, and a sodium/creatinine ratio (Na/Cre ratio) indicates a ratio of a sodium ion concentration in urine to a creatinine concentration in urine.

Sodium ions are contained in, for example, salt, potassium ions are contained in, for example, vegetables and fruits, magnesium ions are contained in, for example, seeds, seaweed, and dairy products, and urea-nitrogen is contained in proteins. Also, the creatinine concentration in urine reflects a muscle mass of the subject and the creatinine is excreted according to the body (physical) condition of the subject. Therefore, by using a nutritional component index obtained by dividing the nutritional component concentration by the creatinine concentration, it is possible to use it as an accurate index that reflects the physical condition of the subject.

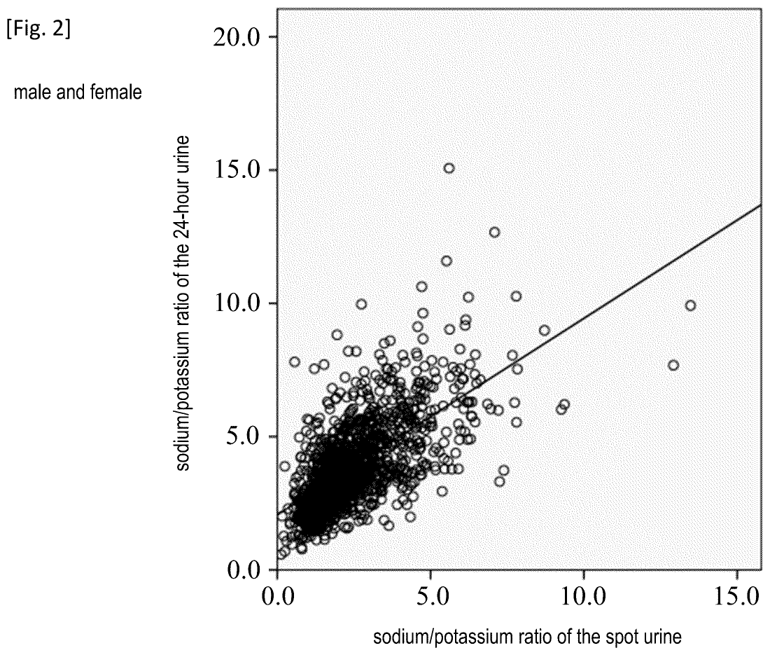

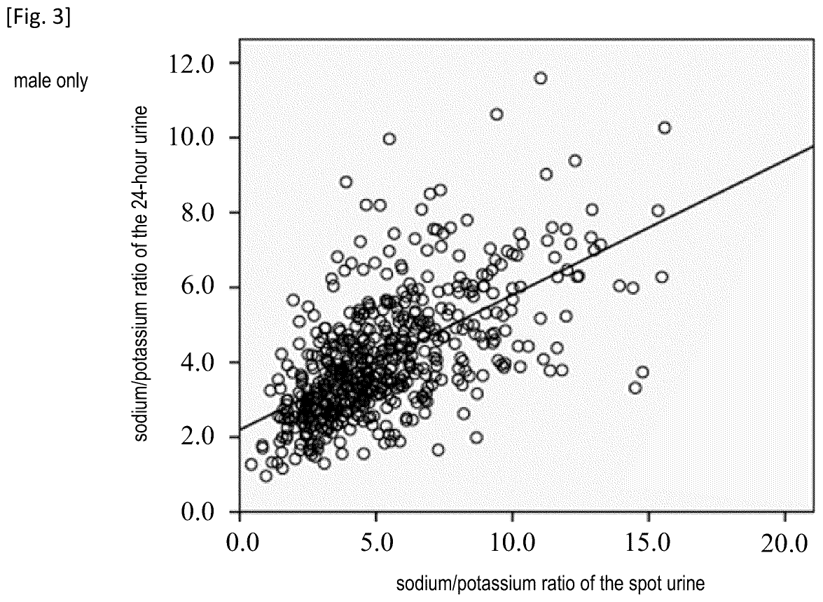

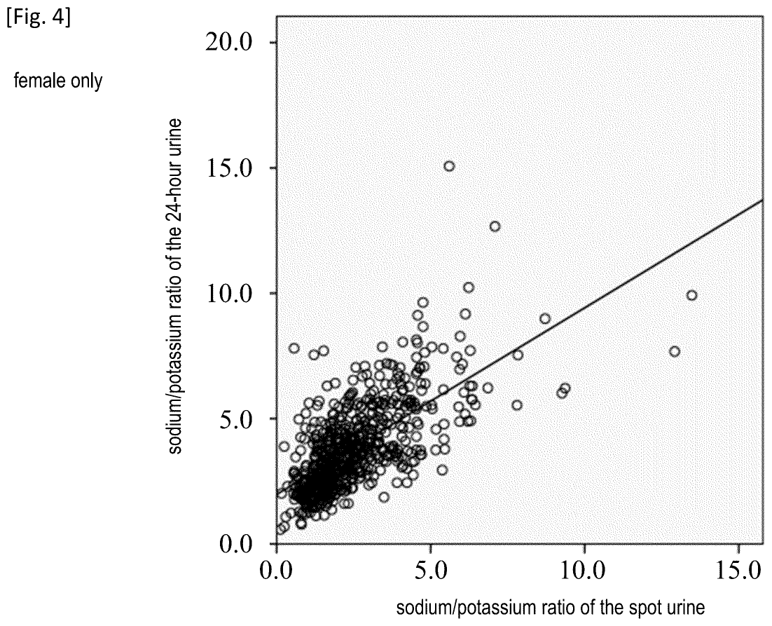

As shown in FIG. 1, it can be understood that the sodium/potassium ratio of the 24-hour urine nutritional component index correlates with the sodium/potassium ratio of the spot urine nutritional component index. Specifically, as shown in FIG. 2, the spot urine sodium/potassium ratio has a linear relationship with the 24-hour urine sodium/potassium ratio. Furthermore, if the subjects are male only or if the subjects are female only, as shown in FIGS. 3-4, the correlation may be better.

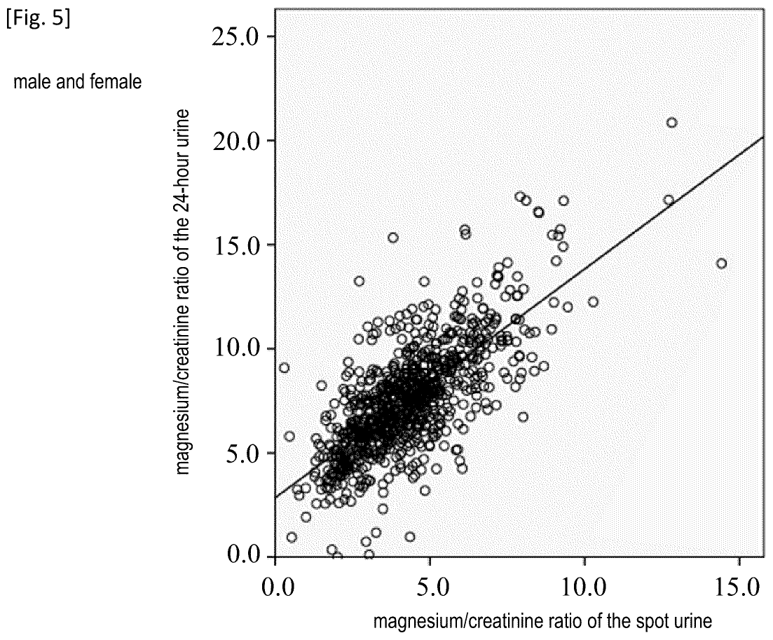

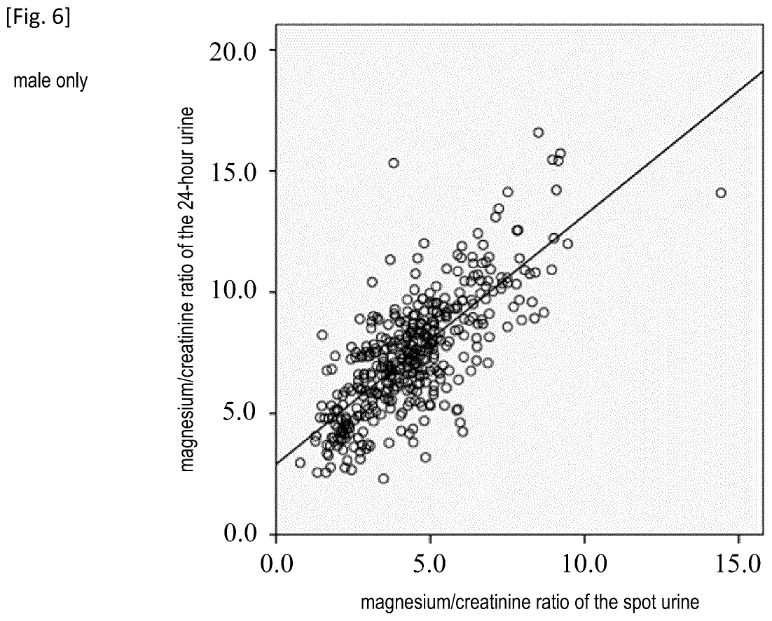

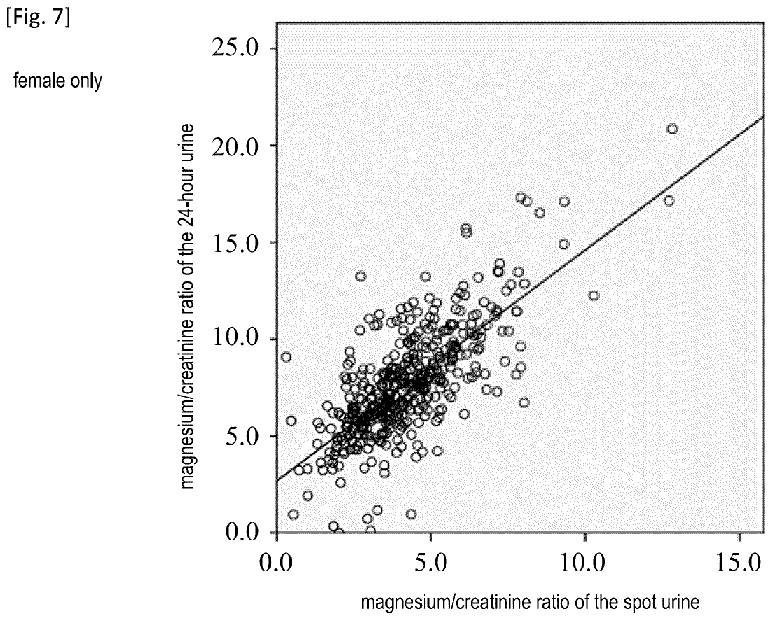

The magnesium/creatinine ratio of the 24-hour urine nutritional component index correlates with the magnesium/creatinine ratio of the spot urine nutritional component index, and as shown in FIG. 5, the magnesium/creatinine ratio of the spot urine nutritional component index has a linear relationship to the magnesium/creatinine ratio. Similarly, if the subjects are male only or if the subjects are female only, as shown in FIGS. 6-7, the correlation may be better.

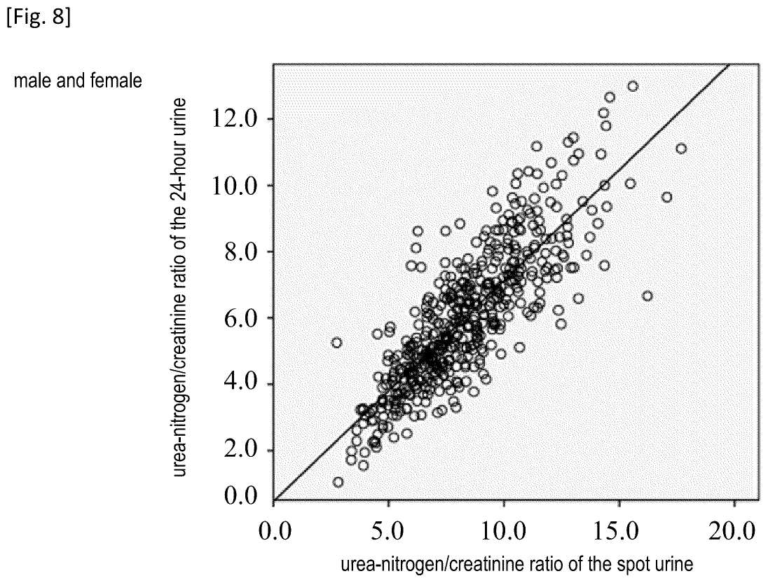

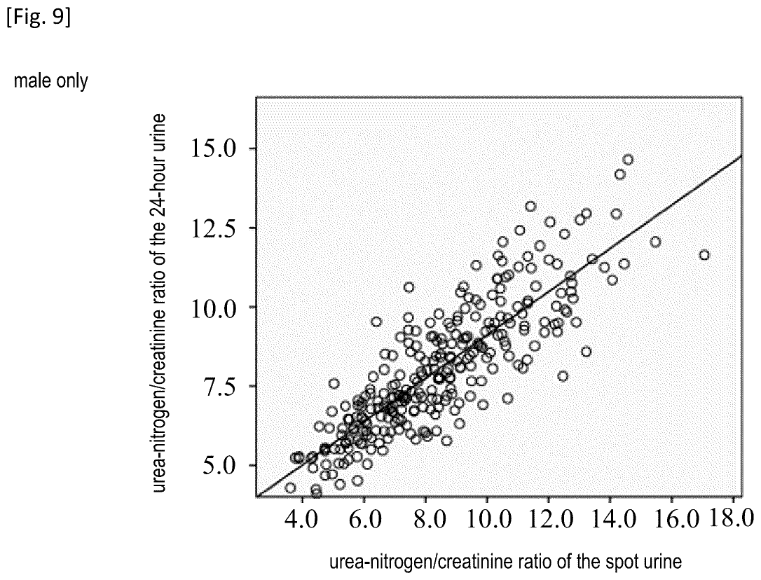

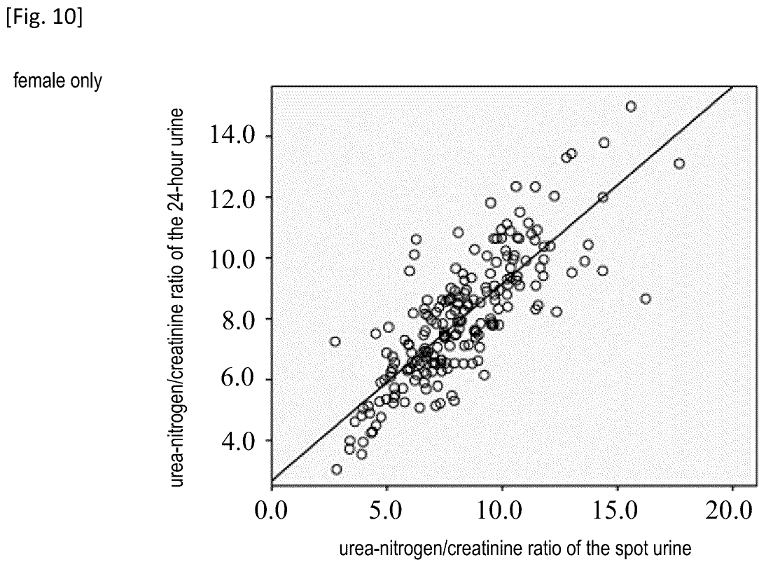

The urea-nitrogen/creatinine ratio of the 24-hour urine nutrient index correlates with the urea-nitrogen/creatinine ratio of the spot urine nutritional component index and has a linear relationship with the urea-nitrogen/creatinine ratio of the 24-hour urine, as shown in FIG. 8. Similarly, if the subjects are only male or when the subjects are only female, as shown in FIGS. 9-10, the correlation may be better.

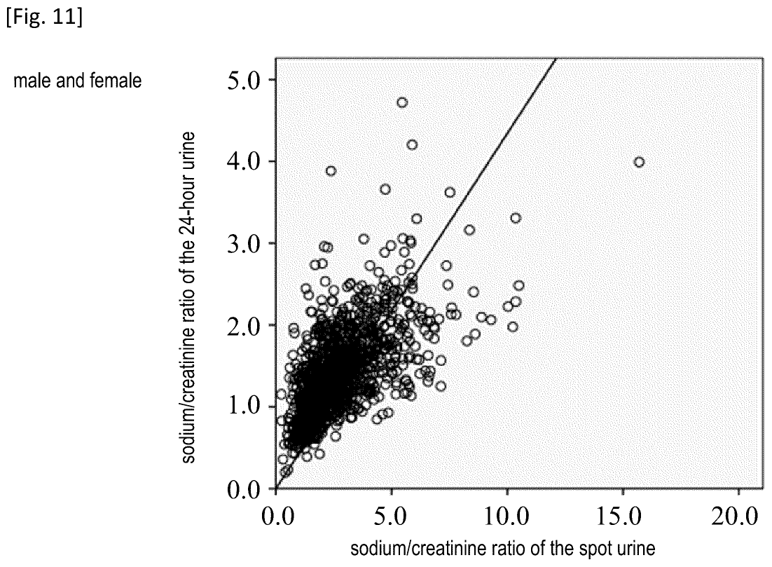

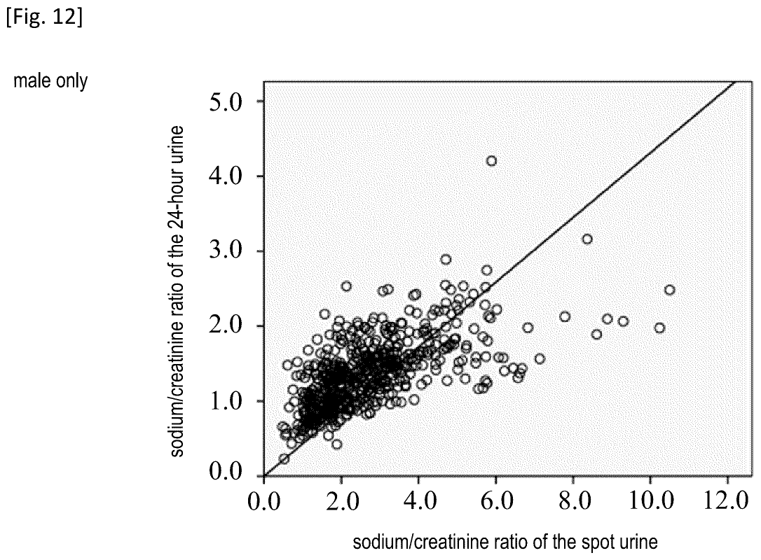

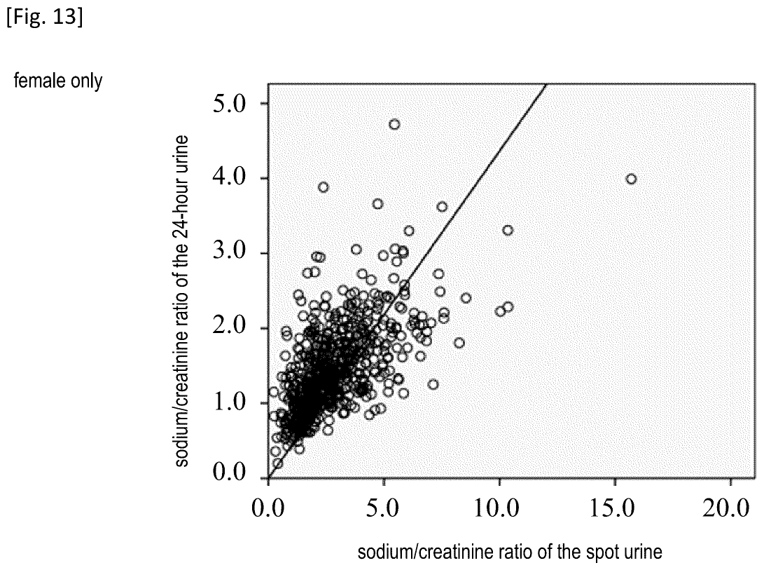

The sodium/creatinine ratio of the 24-hour urine nutrient index correlates with the sodium/creatinine ratio of the spot urine nutritional index and has a linear relationship with the sodium/creatinine ratio of the 24-hour urine, as shown in FIG. 11. Similarly, if the subjects are only male or if the subjects are only female, as shown in FIGS. 12-13, the correlation may be better.

In other words, by evaluating the sodium/potassium ratio, the magnesium/creatinine ratio, the urea-nitrogen/creatinine ratio, and the sodium/creatinine ratio in the spot urine nutritional component indexes, it is possible to evaluate equivalent to an evaluation of the 24-hour urine nutritional component index corresponding to those.

Here, since each of the spot urine nutritional component indexes independently correlates with the 24-hour urine nutritional component index, by selecting two or more of nutritional component indexes from the sodium/potassium ratio, the magnesium/creatinine ratio, the urea-nitrogen/creatinine ratio, and the sodium/creatinine ratio, it is possible to evaluate equivalent to an evaluation of the 24-hour urine nutritional component index from the spot urine nutritional component indexes of the selected nutritional component indexes.

Similarly, each of the spot urine nutrient indexes of the magnesium/creatinine ratio, the urea-nitrogen/creatinine ratio, and the sodium/creatinine ratio independently correlates with the 24-hour urine nutritional component index. By adopting spot urine nutritional component indexes indicating a ratio of each of nutritional component concentrations two or more selected from magnesium ion concentration, urea-nitrogen concentration, and sodium ion concentration, the nutritional component concentration contained in one spot urine excreted by a human, to a creatinine concentration in the one spot urine, it is possible to evaluate equivalent to an evaluation of the 24-hour urine nutritional component index from the adopted spot urine nutritional component index.

Therefore, in order to specifically evaluate the nutritional status of the human, the present invention uses four types of the spot urine nutritional component indexes of the sodium/potassium ratio, the magnesium/creatinine ratio, the urea-nitrogen/creatinine ratio, and the sodium/creatinine ratio as basic indexes, and constitutes a dietary habit evaluation system and a dietary habit evaluation method. By using these indexes, it is possible to see the overall balance of the dietary habit of the human by using one spot urine of the human.

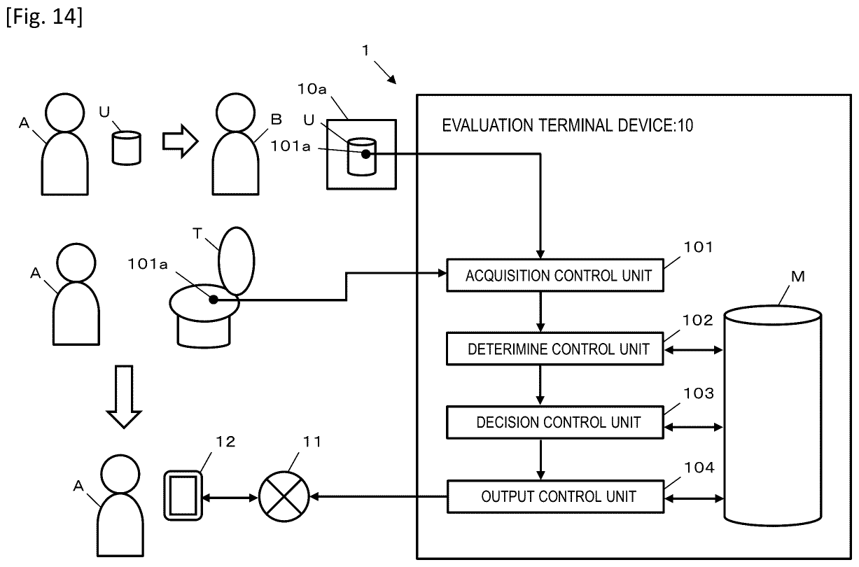

First, a dietary habit evaluation system 1 according to the present invention includes, for example, as shown in FIG. 14, an evaluation terminal device 10, a network 11, and a user terminal device 12.

The evaluation terminal device 10 is a general computer used by an evaluator B who evaluates the urine of a human (subject) A, decides a diet evaluation pattern of the human A based on the spot urine nutritional component indexes of the spot urine of human A, and outputs diet evaluation comments (evaluation contents) corresponding to the diet evaluation pattern. Also, the evaluation terminal device 10 may be a server on the network in order to process data.

The network 11 communicably connects with the evaluation terminal device 10 and the user terminal device 12. The network 11 includes wireless communication networks such as a LAN (Local Area Network) via a Wifi (registered trademark) access point, a WAN (Wide Area Network) via a wireless base station, a third generation (3G) communication system, 4th generation (4G) communication system, 5th generation (5G) and later communication systems, Bluetooth (registered trademark), and specified low power radio system.

The user terminal device 12 is a general computer used by the human A, and includes, for example, a mobile terminal device with a touch panel, a desktop terminal device, a tablet terminal device, and a wearable terminal device.

The evaluation terminal device 10 and the user terminal device 12 include an input unit for inputting instructions, a storage unit for storing information, and an output unit for outputting information. The evaluation terminal device 10 and the user terminal device 12 have a built-in CPU, ROM, RAM, etc. (not shown), and the CPU uses the RAM as a work area, and executes program stored in the ROM or the like. Further, each control unit described later also realizes a function of each control unit by the CPU executing the program.

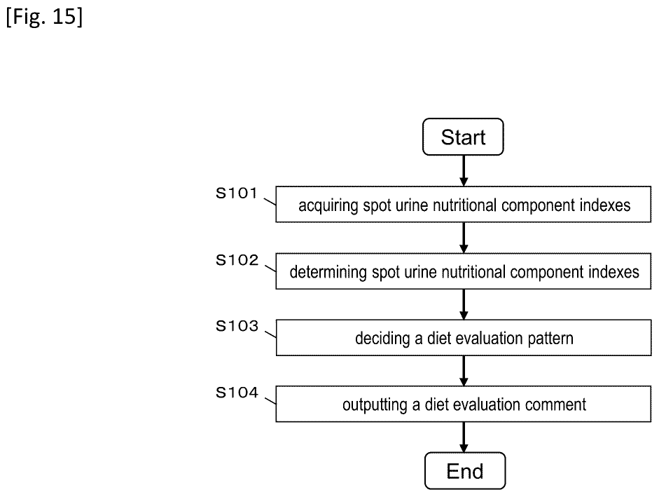

Next, as shown in FIG. 15, the configuration and execution procedure for the embodiment of the invention will be described. First, the evaluator B collects one spot urine excreted by the human A in order to evaluate the dietary habit of the human A.

The type of one spot urine U of human A is not particularly limited. For example, the one spot urine U may be a spot urine U excreted first by the human A in the morning after waking (getting) up, a spot urine U excreted first by the human A in the afternoon after waking up, or the like.

The evaluator B processes the spot urine U, and an acquisition control unit 101 of the evaluation terminal device 10 acquires spot urine nutritional component indexes related to nutritional components including a sodium/potassium ratio, a magnesium/creatinine ratio, a urea-nitrogen/creatinine ratio, and a sodium/creatinine ratio in the spot urine (FIG. 2: S101).

Here, the acquisition method of the acquisition control unit 101 is not particularly limited. For example, when evaluating the dietary habit of human A at a dietary habit evaluation center such as a pharmacy, school medical examination (kindergarten, elementary school, junior high school, high school, university), company medical examination, medical examination center, etc., the evaluator B obtains the spot urine U of the human A and stores it in a urine analysis unit 10a of the evaluation terminal device 10. The urine analysis unit 10a is provided with a sensor 101a that measures the concentration of the nutritional component in the spot urine U. When the sensor 101a contacts with the spot urine U, the sensor 101a outputs a measurement signal, and the acquisition control unit 101 acquires nutritional component concentration in the spot urine U based on the measurement signal from the sensor 101a.

The type of sensor 101a is set appropriately according to the type of spot urine nutritional component index. For example, when the spot urine nutritional component indexes are the sodium/potassium ratio, the magnesium/creatinine ratio, the urea-nitrogen/creatinine ratio, and the sodium/creatinine ratio, the sensor 101a is used by ion-selective electrode methods corresponding to sodium ion concentration, potassium ion concentration, magnesium ion concentration, and urea-nitrogen concentration, and reagent method, enzymatic method, spectrophotometric method, and high performance chromatography, corresponding to creatinine concentration.

Further, when a toilet bowl T is provided with the sensor 101a, when the human A urinates, the sensor 101a outputs a measurement signal by contacting with the spot urine U, and an acquisition control unit 101 acquires nutritional component concentration in the spot urine U based on the measurement signal from the sensor 101a.

Then, the acquisition control unit 101 calculates and acquires spot urine nutritional component index using the acquired nutritional component concentration. Here, if there are four types of the spot urine nutritional component indexes of the sodium/potassium ratio, the magnesium/creatinine ratio, the urea-nitrogen/creatinine ratio, and the sodium/creatinine ratio, the acquisition control unit 101 calculates the sodium/potassium ratio based on the sodium ion concentration and the potassium ion concentration, calculates magnesium/creatinine ratio based on the magnesium ion concentration and the creatinine concentration, calculates the urea-nitrogen/creatinine ratio based on the urea-nitrogen concentration and the creatinine concentration, and calculates the sodium/creatinine ratio based on the sodium ion concentration and the creatinine concentration.

As another embodiment, when the evaluator B acquires the spot urine nutritional component index of the spot urine U of the human A at an external measuring institution, the evaluator B inputs the acquired spot urine nutritional component index into the evaluation terminal device 10, and the acquisition control unit 101 acquires the spot urine nutritional component index in the spot urine U.

Now, when the acquisition of the spot urine nutritional component indexes is completed, a determination control unit 102 of the evaluation terminal device 10 compares the acquired spot urine nutritional component index with a reference value preset for the spot urine nutritional component index for each of the spot urine nutritional component indexes, and determines whether or not the spot urine nutritional component index is equal to or more than the reference value for each of the spot urine nutritional component indexes (FIG. 2: S102)

Here, the determination method of the determination control unit 102 is not particularly limited. For example, the determination method is appropriately changed depending on the type of the spot urine nutritional component index. For example, if the spot urine nutritional component index is the sodium/potassium ratio, it corresponds to the sodium/potassium ratio of the 24-hour urine nutritional component index. It is determined that the lower the sodium/potassium ratio, the less the intake of salt and the higher the intake of vegetables and fruits consumed (ate) in the diet of human A. Therefore, when the acquired sodium/potassium ratio is less than the reference value, the determination control unit 102 determines that the sodium/potassium ratio is appropriate, and when the sodium/potassium ratio is the reference value or more, the determination control unit 102 determines that the sodium/potassium ratio is inappropriate.

The reference value of the sodium/potassium ratio of the one spot urine may be preferably determined based on a desired daily intake of sodium (or salt intake) and a desired daily intake of potassium of a human.

When the spot urine nutritional component index is the magnesium/creatinine ratio, it corresponds to the magnesium/creatinine ratio of the 24-hour urine nutritional component index. It is determined that the higher the magnesium/creatinine ratio, the higher the intake of seaweed, seeds, and dairy products consumed in the diet of human A. Therefore, when the acquired magnesium/creatinine ratio is the reference value or more, the determination control unit 102 determines that the magnesium/creatinine ratio is appropriate. When the acquired magnesium/creatinine ratio is less than the reference value, the determination control unit 102 determines that the magnesium/creatinine ratio is inappropriate.

The reference value of the magnesium/creatinine ratio of one spot urine may preferably be determined based on a desired daily intake of magnesium for a human corresponding to the weight of the human.

When the spot urine nutritional component index is the urea-nitrogen/creatinine ratio, it corresponds to the urea-nitrogen/creatinine ratio of the 24-hour urine nutritional component index. It is determined that the higher the urea-nitrogen/creatinine ratio, the higher the intake of meat, seafood, eggs, and soybeans consumed in the diet of human A. Therefore, when the acquired urea-nitrogen/creatinine ratio is the reference value or more, the determination control unit 102 determines that the urea-nitrogen/creatinine ratio is appropriate, When the acquired the urea-nitrogen/creatinine ratio is less than the reference value, the determination control unit 102 determines that urea-nitrogen/creatinine ratio is inappropriate.

The reference value of the urea-nitrogen/creatinine ratio of one spot urine may preferably be determined based on a desired daily intake of protein for a human corresponding to the weight of the human.

When the spot urine nutritional component index is the sodium/creatinine ratio, it corresponds to the sodium/creatinine ratio of the 24-hour urine nutritional component index. It is determined that the lower the sodium/creatinine ratio, the less the intake of salt consumed in the diet of human A. Therefore, when the acquired sodium/creatinine ratio is less than the reference value, the determination control unit 102 determines that the sodium/creatinine ratio is appropriate. When the sodium/creatinine ratio is the reference value or more, the determination control unit 102 determines that the sodium/creatinine ratio is inappropriate.

The reference value of the sodium/creatinine ratio of one spot urine may preferably be determined based on a desired daily sodium intake (or salt intake) of a human corresponding to the body weight of the human. In this way, the determination control unit 102 determines whether or not the spot urine nutritional component index is an appropriate determination or an inappropriate determination for each of the spot urine nutritional component indexes based on the determination result of whether or not the spot urine nutritional component index is the reference value or more.

In addition, the reference value for each of the types of the spot urine nutritional component indexes is stored in advance in the memory M of the evaluation terminal device 10. The determination control unit 102 acquires the reference value corresponding to the type of spot urine nutritional component index from the memory M, and uses for the determination.

Also, the reference value may be appropriately changed according to the physical information (sex, age, height, weight, etc.) of the human A in addition to the type of the spot urine nutritional component index. If the spot urine nutritional component indexes are the sodium/potassium ratio, the magnesium/creatinine ratio, the urea-nitrogen/creatinine ratio, or the sodium/creatinine ratio, the reference values may vary slightly depending on sex and age of the human A and Body Mass Index (BMI) {weight (kg)/height (m)2}. For example, as shown in FIG. 1, in the sodium/potassium ratio, the magnesium/creatinine ratio, the urea-nitrogen/creatinine ratio, and the sodium/creatinine ratio, the correlation coefficient may be better by dividing them according to sex of the subject.

Therefore, the determination control unit 102 acquires body information including sex, age, or BMI of the human A from the human A or the evaluator B, acquires a reference value corresponding to a combination of the type of the acquired spot urine nutritional component index and the acquired body information, and determines whether or not the acquired spot urine nutritional component index is the acquired reference value or more. In this way, by changing the reference value according to the type of spot urine nutritional component index and the body information of the human A, it is possible to make a determination appropriate for the body information of the human A, and to evaluate the dietary habit of the human A with high accuracy by using one spot urine. As the types of body information increase, it is possible to set more detailed reference values. Therefore, by adding sex, age, and BMI to the body information, it is possible to evaluate the dietary habit of the human A with higher accuracy.

When the determinations of the spot urine nutritional component indexes are completed, a decision control unit 103 of the evaluation terminal device 10 decides a diet evaluation pattern indicating a balance of the diet of the human A from the determination result for each of the determined spot urine nutritional component indexes (FIG. 2: S103).

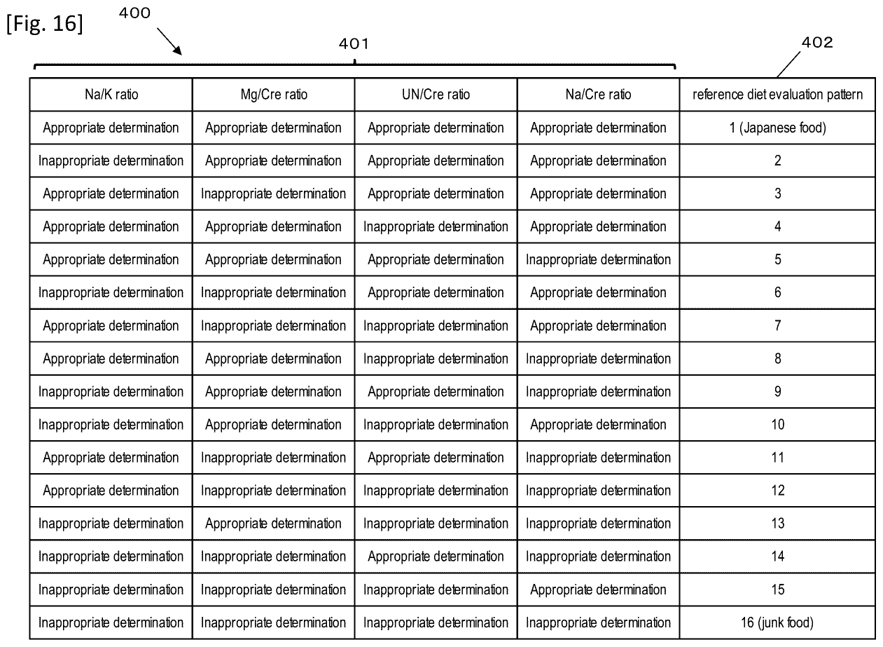

Here, the decision method of the decision control unit 103 is not particularly limited. For example, the decision control unit 103 refers to a pattern sheet stored in the memory M in advance. As shown in FIG. 16, the pattern sheet 400 stores reference determination results 401 for each of the spot urine nutritional component indexes in association with reference diet evaluation patterns 402 specified by a combination of the reference determination results 401.

In the reference determination results 401, an appropriate determination or an inappropriate determination is set for each of the spot urine nutritional component indexes (for example, the sodium/potassium ratio, the magnesium/creatinine ratio, the urea-nitrogen/creatinine ratio, the sodium/creatinine ratio).

The number of the reference diet evaluation patterns 402 is 2 4=16 obtained by taking the number (2) of the types (an appropriate determination/an inappropriate determination) of the reference determination results 401 as the base and the number (4) of the spot urine nutrient component index as the power number (exponent).

Here, when the reference determination results 401 of the sodium/potassium ratio, the magnesium/creatinine ratio, the urea-nitrogen/creatinine ratio, and the sodium/creatinine ratio are all appropriate determinations, the reference diet evaluation pattern 402 is set to “1” which is the best dietary balance. When the reference diet evaluation pattern is “1”, for example, it is named “Japanese food”. Further, when the reference determination result 401 of the sodium/potassium ratio is an inappropriate determination, and the reference determination results 401 of the magnesium/creatinine ratio, the urea-nitrogen/creatinine ratio, and the sodium/creatinine ratio are appropriate determinations, the reference diet evaluation pattern 402 is set to “2” which is a good diet balance, but showing a lack of vegetables. On the other hand, when the reference determination results 401 of the sodium/potassium ratio, the magnesium/creatinine ratio, the urea-nitrogen/creatinine ratio, and the sodium/creatinine ratio are all inappropriate determinations, the reference diet evaluation pattern 402 is set to “16” which is the worst diet balance. If the reference diet evaluation pattern is “16”, for example, it is named “junk food”.

In this way, a combination of the appropriate determination or the inappropriate determination of 4 types of the spot urine nutritional component indexes forms 16 types of diet evaluation patterns. In other words, plural reference diet evaluation patterns are formed by combining the reference determination results of the appropriate determination or the inappropriate determination for each of the spot urine nutritional component indexes. Since each of these reference diet evaluation patterns requires different nutritional components, it will be necessary to provide the most effective comment for each of the diet evaluation patterns.

Now, the decision control unit 103 decides a reference diet evaluation pattern 402 of a combination of the reference determination results 401 matching each of the determination results for each of the determined spot urine nutritional component indexes in plural reference diet evaluation patterns 402 as a diet evaluation pattern of the human A. Specifically, the decision control unit 103 matches the determined determination result with the reference determination result 401 of the pattern sheet 400 for each of the spot urine nutritional component indexes, and decides the reference diet evaluation pattern 402 of the pattern sheet 400 when each of the determination results for each of the spot urine nutritional component indexes matches each of the reference determination results 401 for each of the spot urine nutritional component indexes as a diet evaluation pattern of the human A. In this way, it is possible to uniquely decide a diet evaluation pattern specified by a combination of determination results for each of the spot urine nutritional component indexes.

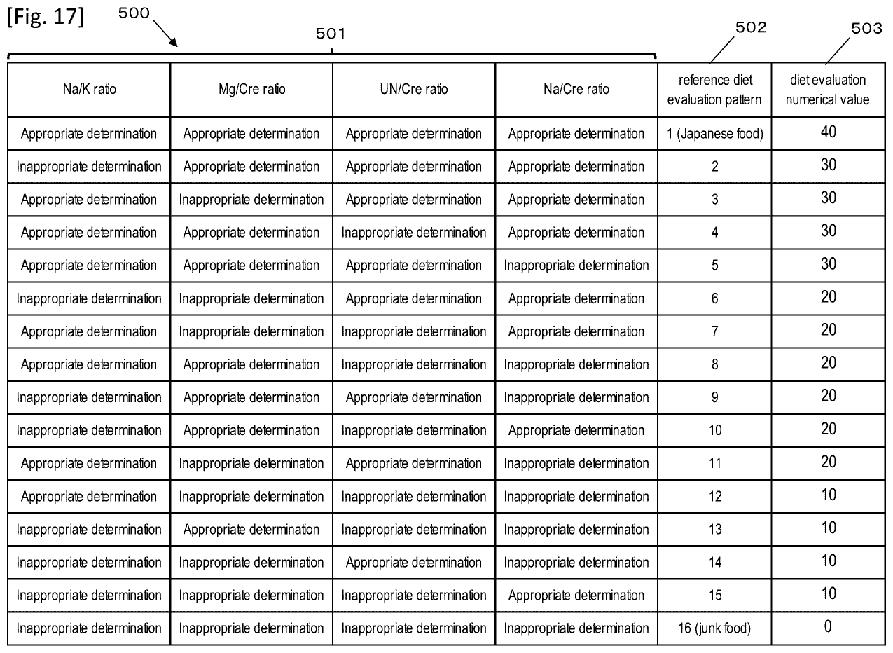

Here, in addition to the diet evaluation pattern, a numerical value indicating a good or bad of the diet evaluation pattern may be added. For example, as shown in FIG. 17, a pattern sheet 500 stores a diet evaluation numerical value 503 indicating a good or bad of the diet evaluation pattern 502 in association with reference determination results 501 for each of the spot urine nutritional component indexes and reference diet evaluation patterns 502.

Then, 10 points are given to the appropriate determination in the reference determination results 501 of the pattern sheet 500, 0 point is given to the inappropriate determinations, and the sum of the points of the reference determination results 501 for each of the spot urine nutritional component indexes is reflected in the diet evaluation numerical value 503. In other words, the diet evaluation numerical value 503 is set to a higher value the more appropriate determinations of the reference determination results 501 of the reference diet evaluation pattern 502. For example, when the reference diet evaluation pattern 502 is “1 (Japanese food)”, the numerical value 503 is “40 points”, and when the reference diet evaluation pattern 502 is “2”, the numerical value 503 is “30 points”, when the reference diet evaluation pattern 502 is “16 (junk food)”, the numerical value 503 is “0 point”. The decision control unit 103 decides the reference diet evaluation pattern 502 and acquires the diet evaluation numerical value 503 corresponding to the reference diet evaluation pattern 502. In this way, the balance of the diet of the human A can be visualized by the diet evaluation pattern as well as the diet evaluation numerical value, so that an intuitive and easy-to-understand evaluation can be presented.

Now, when the decision of the diet evaluation pattern of human A is completed, an output control unit 104 of the evaluation terminal device 10 outputs a diet evaluation comment improving the balance of the diet of the human A for the diet evaluation pattern (FIG. 2: S104).

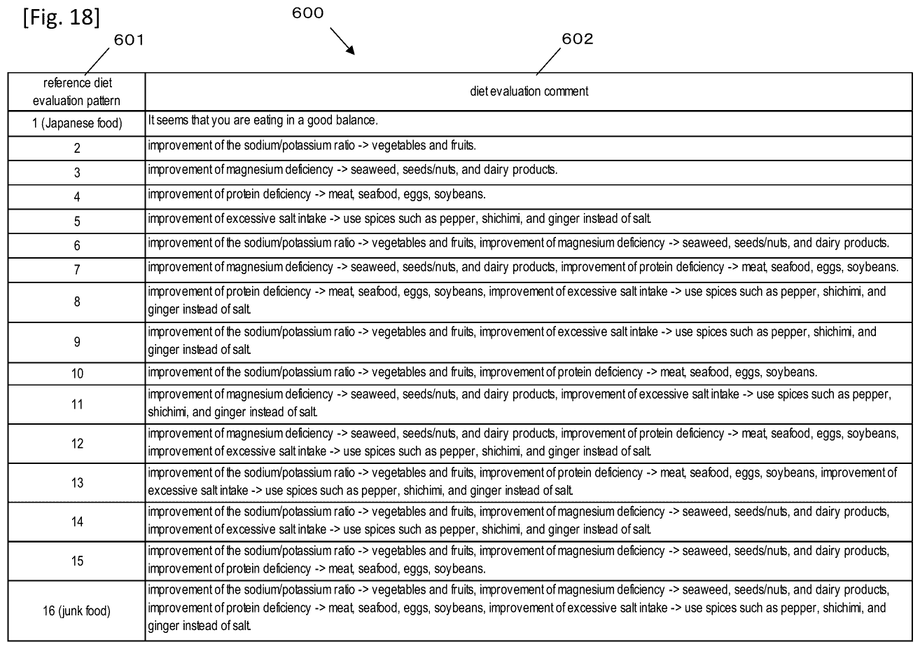

Here, the output method of the output control unit 104 is not particularly limited. For example, the output control unit 104 refers to a comment sheet stored in the memory M in advance. As shown in FIG. 18, the comment sheet 600 stores reference diet evaluation patterns 601 in association with diet evaluation comments 602 that improves the balance of the diet for the reference diet evaluation pattern 601.

For example, when the reference diet evaluation pattern 601 is “1 (Japanese food)”, the diet evaluation comment 602 is a comment for maintaining the current diet, such as “It seems that you are eating in a good balance.” Further, when the reference diet evaluation pattern 601 is “2”, the diet evaluation comment 602 is a comment that lists necessary foods to improve the sodium/potassium ratio, such as “improvement of the sodium/potassium ratio->vegetables and fruits.”

In addition, when the reference diet evaluation pattern 601 is “3”, the diet evaluation comment 602 is a comment that lists necessary foods to improve the magnesium/creatinine ratio, such as “improvement of magnesium deficiency->seaweed, seeds/nuts, and dairy products”. When the reference diet evaluation pattern 601 is “4”, the diet evaluation comment 602 is a comment that lists necessary foods to improve the urea-nitrogen/creatinine ratio, such as “improvement of protein deficiency->meat, seafood, eggs, soybeans”. When the reference diet evaluation pattern 601 is “5”, the diet evaluation comment 602 is a comment that lists necessary foods to improve sodium/creatinine, such as “improvement of excessive salt intake->use spices such as pepper, shichimi, and ginger instead of salt.”

Further, when the reference diet evaluation pattern 601 is “16 (junk food)”, the reference diet evaluation pattern 601 indicates that all the spot urine nutritional component indexes are inappropriate determinations, the diet evaluation comment 602 is a combination of the inappropriate determination comments corresponding to the inappropriate determination of each of the spot urine nutritional component indexes. For example, the diet evaluation comment 602 is a comment on improving the sodium/potassium ratio, magnesium/creatinine ratio, urea-nitrogen/creatinine ratio and sodium/creatinine ratio, such as “improvement of the sodium/potassium ratio->vegetables and fruits, improvement of magnesium deficiency->seaweed, seeds and nuts, dairy products, improvement of protein deficiency->meat, seafood, eggs, soybeans, improvement of excessive salt intake->use spices such as pepper, shichimi, and ginger instead of salt.”

Thus, in the diet evaluation comment 602, comments for improving the current diet of human A are set according to the type of the reference diet evaluation pattern 601. In this way, the human A can confirm his/her diet evaluation pattern and comment, and it is possible to make use for the review of the dietary habit of oneself. In addition, by using the spot urine nutritional component indexes described above, it is possible to evaluate the diet of human A as a whole, taking into consideration the balance of the diet of human A.

In addition, the diet evaluation comment 602 of this comprises as the diet evaluation comment by acquiring the inappropriate determination comment corresponding to the spot urine nutritional component index of the inappropriate determination for the reference diet evaluation pattern with the inappropriate determination for each spot urine nutritional component index with the inappropriate determination, and by adding the acquired inappropriate determination comments. In practice, nutritional components required for each of the diet evaluation comments are different, so the diet evaluation comments for each of the diet evaluation patterns are appropriately designed and changed.

Now, the output control unit 104 compares the decided diet evaluation pattern with the reference diet evaluation pattern 601 of the comment sheet 600, and acquires the diet evaluation comment 602 of the comment sheet 600 when the diet evaluation pattern matches the reference diet evaluation pattern 601, and outputs it. In this way, it possible to easily evaluate the balance of the diet of the human A using one spot urine of the human A. When the diet evaluation numerical values are set in the diet evaluation patterns, the output control unit 104 may output the diet evaluation numerical value together with the diet evaluation pattern and the diet evaluation comment 602.

Here, the diet evaluation comment 602 may be printed on a predetermined sheet at the dietary evaluation center by the evaluator B, and handed directly to the human A to give advice on dietary support advice, or in any other configuration.

For example, when the human A accesses the evaluation terminal device 10 via the network 11 using the user terminal device 12 and inputs the identification information of the human A to the evaluation terminal device 10, the output control unit 104 of the evaluation terminal device 10 may be configured to make the user terminal device 12 display the diet evaluation comment 602 corresponding to the spot urine U of the human A.

In the embodiment of the present invention, 4 types of the sodium/potassium ratio, the magnesium/creatinine ratio, the urea-nitrogen/creatinine ratio, and the sodium/creatinine ratio are used as the spot urine nutritional component indexes, other indexes may be added. For example, as the spot urine nutritional component indexes, the concentration of nutritional components contained (included) in spot urine such as minerals, for example, zinc, calcium, chromium, selenium, iron, copper, manganese, molybdenum, iodine, phosphorus except sodium, potassium, and magnesium, vitamins, for example, vitamin A, vitamin B group, vitamin C, vitamin D, vitamin E, and vitamin K, and a ratio of the nutritional component concentration to creatinine concentration can be mentioned.

In addition, in the embodiment of the present invention, the sodium/creatinine ratio is used as one spot urine nutritional component index as a guide to salt (salt) intake, instead it, a spot urine nutritional component index based on the sodium ion concentration in one spot urine may be used. For example, the acquisition control unit 101 calculates a sodium ion concentration contained in 24-hour urine from the sodium ion concentration contained in one spot urine excreted by a human, and calculates a sodium ion concentration contained in 24-hour urine. And the sodium ion concentration contained in 24-hour urine is adopted to the daily salt intake of the human as a spot urine nutritional component index. A preferable reference value for salt intake per day is, for example, the International reference of 5.0 g, the Japanese reference of 8.0 g for male, and 7.0 g for female. By using this, it is easy to set reference value and it is easy for the subject to understand.

Further, the embodiment of the present invention is configured to decide a diet evaluation pattern by evaluating spot urine nutrient component indexes and to output a diet evaluation comment matching this diet evaluation pattern. The present invention may add BMI evaluation or questionnaire evaluation in addition to the diet evaluation pattern.

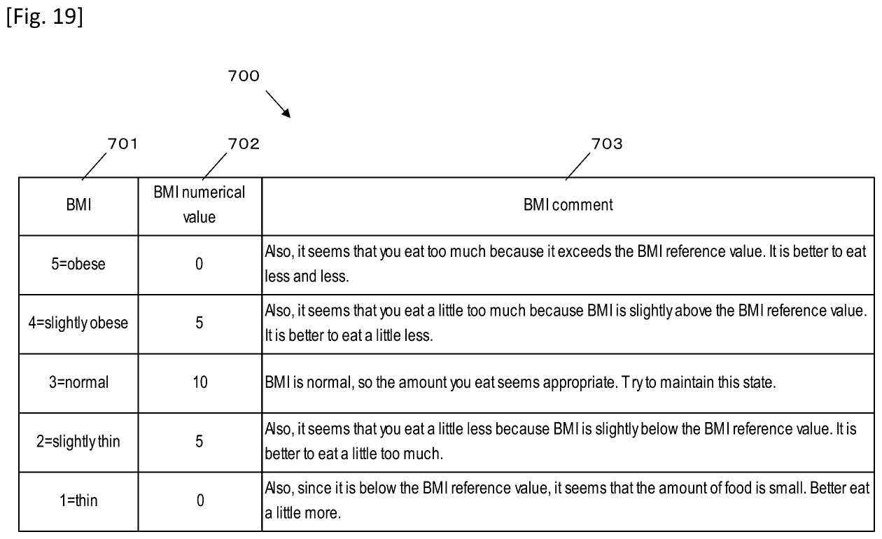

For example, when the output control unit 104 outputs the diet evaluation numerical value corresponding to the diet evaluation pattern, a BMI control unit of the evaluation terminal device 10 receives an input of a BMI of the human A from the human A or the evaluator B and refers to a BMI sheet stored in advance in the memory M. As shown in FIG. 19, the BMI sheet 700 stores classified BMIs 701, BMI numerical values 702 indicating a good or bad of the BMI in association with BMI comments 703 for improving diet habit according to the BMI.

Here, when the BMI 701 is “obese” or “thin”, the BMI value 702 is “0 point”, and when the BMI 701 is “slightly obese” or “slightly thin”, the BMI value 702 is “5 points”, and when the BMI 701 is “normal”, the BMI numerical value 702 is “10 points”. That is, the BMI numerical value 702 is set to a higher value as the BMI is closer to normal.

Then, the BMI control unit compares the input BMI of the human A with the BMI 701 of the BMI sheet 700, acquires the BMI numerical value 702 and the BMI comment 703 of the BMI sheet 700 when the BMI of the human A matches the BMI 701. The output control unit 104 calculates a total value by adding the BMI numerical value 702 acquired by the BMI control unit to the diet evaluation numerical value corresponding to the diet evaluation pattern, and outputs the total value, the diet evaluation comment corresponding to the diet evaluation pattern, and the BMI comment 703 corresponding to the BMI 701. In this way, it is possible to comprehensively determine the diet habit and body type by numerical value, and to output the BMI comment corresponding to the BMI.

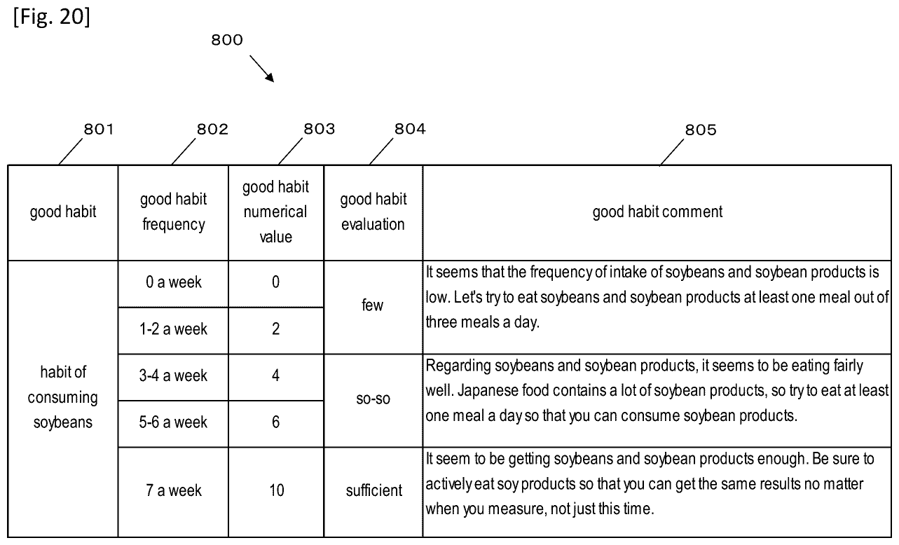

For example, when the output control unit 104 outputs a diet evaluation numerical value corresponding to a diet evaluation pattern, the evaluator B obtains a questionnaire regarding the frequency of good habit from the human A. The good habit may be a habit of consuming soybeans, a habit of consuming seafood, and a habit of exercising, etc. When the good habit is the habit of consuming soybeans, or the habit of consuming seafood, a questionnaire of the frequency of the good habit is obtained by getting the human A to answer how often he (or she) consumes them in a week. When the good habit is the habit of exercising, the human A is asked to respond how often he/she exercises them in a week. A questionnaire control unit of the evaluation terminal device 10 receives an input of the questionnaire result of the human A from the human A or the evaluator B, and refers to a good habit questionnaire sheet stored in the memory M in advance. As shown in FIG. 20, the good habit questionnaire sheet 800 stores a good habit 801, a good habit frequency 802, and a good habit numerical value 803 corresponding to the good habit frequency 802 in association with a good habit evaluation 804 corresponding to the good habit numerical value 803, and a good habit comment 805 corresponding to the good habit numerical value 803. The good habit frequency 802 is, for example, 0 a week, 1-2 a week, 3-4 a week, 5-6 a week, 7 a week, and the good habit numerical value 803 is set to be higher as the good habit frequency 802 is higher.

Then, the questionnaire control unit compares the questionnaire result of the human A with the good habit frequency 802 of the good habit questionnaire sheet 800, and acquires the good habit numerical value 803 of the good habit questionnaire sheet 800 when the questionnaire result of the human A matches the good habit frequency 802, the good habit evaluation 804, and the good habit comments 805. The output control unit 104 calculates a total value by adding the good habit numerical value 803 acquired by the questionnaire control unit to the numerical value corresponding to the diet evaluation pattern, and outputs the total value, the diet evaluation comment corresponding to the diet evaluation pattern, the good habit evaluation 804 corresponding to the good habit questionnaire result, and the good habit comment 805. In this way, it is possible to comprehensively determine diet habit and good habit by numerical value, and to output the good habit comment corresponding to the good habit.

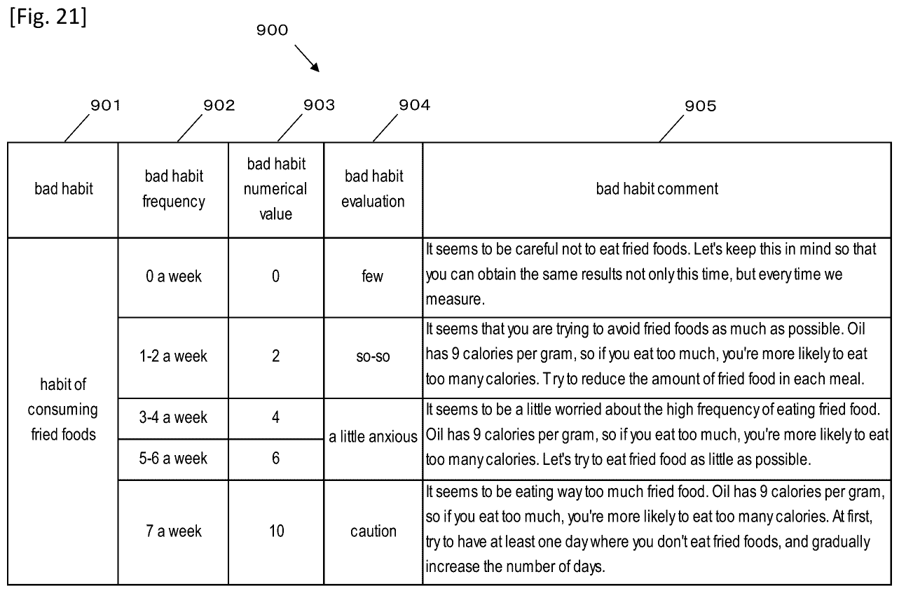

On the other hand, the evaluator B obtains a questionnaire regarding the frequency of bad habit for the human A. The bad habit may be a habit of consuming fried foods, a habit of consuming sweets, etc. When the bad habit is the habit of consuming (eating) fried foods or the habit of consuming sweets, a questionnaire of the frequency of the bad habit is obtained by getting the human A to answer how often he (or she) consumes them in a week. The questionnaire control unit receives an input of the questionnaire result of the human A from the human A or the evaluator B, and refers to a bad habit questionnaire sheet stored in the memory M in advance. As shown in FIG. 21, the bad habit questionnaire sheet 900 stores a bad habit 901, a bad habit frequency 902, a bad habit numerical value 903 corresponding to the bad habit frequency 902, and a bad habit evaluation 904 corresponding to the bad habit numerical value 903 in association with a bad habit comment 905 corresponding to the bad habit numerical value 903. The bad habit frequency 902 is, for example, 0 a week, 1-2 a week, 3-4 a week, 5-6 a week, 7 a week, and the bad habit numerical value 903 is set to be lower as the bad habit frequency 902 is higher.

Then, the questionnaire control unit compares the questionnaire result of human A with the bad habit frequency 902 of the bad habit questionnaire sheet 900, and acquires the bad habit numerical value 903 the bad habit questionnaire sheet 900 when the questionnaire result of the human A matches the bad habit frequency 902, the bad habit evaluation 904, and the bad habit comment 905. The output control unit 104 calculates a total value by adding the bad habit numerical value 903 acquired by the questionnaire control unit to the diet evaluation numerical value corresponding to the diet evaluation pattern, and outputs the total value, the diet evaluation comment corresponding to the diet evaluation pattern, the bad habit evaluation 904 corresponding to the bad habit questionnaire result, and the bad habit comment 905. In this way, it is possible to comprehensively determine diet habit and bad habit by numerical value, and to output the bad habit comment corresponding to the bad habit.

In the above description, the evaluation by BMI and the evaluation by the good or bad habit questionnaire are added to the diet evaluation pattern. The present invention may add the evaluation by BMI and the evaluation by the good and bad habit questionnaire in addition to the diet evaluation pattern.

In the embodiment of the present invention, by using 4 types of spot urine nutritional component indexes of the sodium/potassium ratio, the magnesium/creatinine ratio, the urea-nitrogen/creatinine ratio, and the sodium/creatinine ratio, the present invention prepared 16 diet evaluation patterns and evaluated the dietary habit of the human. The present invention may select two or more spot urine nutritional component indexes, prepare diet evaluation patterns corresponding to the number of indexes selected, and evaluate the dietary habit of the human.

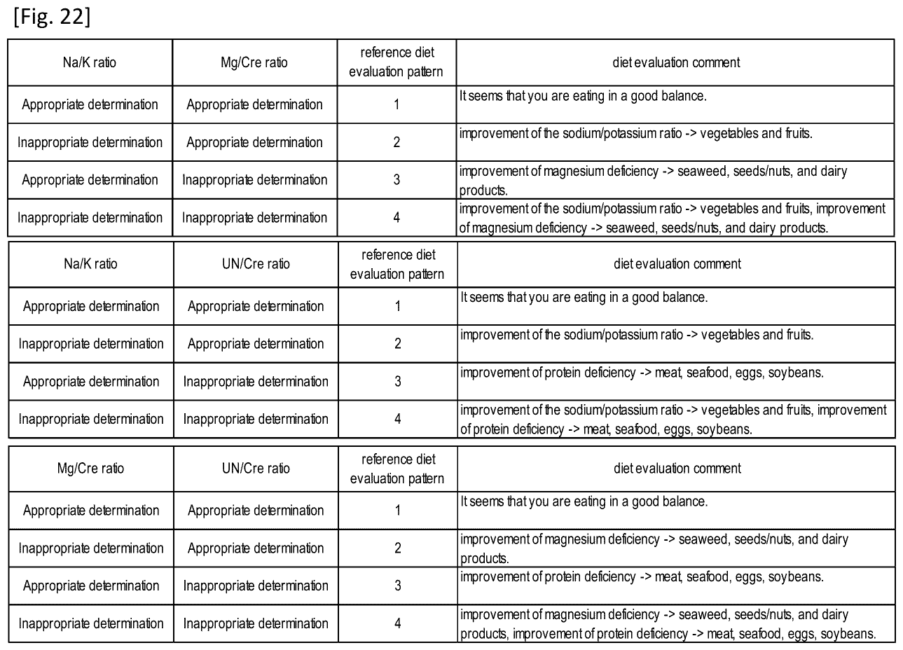

For example, as shown in FIG. 22, when dealing with 2 types of spot urine nutritional component indexes of the sodium/potassium ratio and the magnesium/creatinine ratio, 4 diet evaluation patterns of comments relating to salt, vegetables/fruits, fruits, seaweed, and dairy products may be prepared. Also, when dealing with 2 types of spot urine nutritional component indexes of the sodium/potassium ratio and the urea-nitrogen/creatinine ratio, 4 diet evaluation patterns of comments relating to salt, vegetables/fruits, and protein may be prepared. Furthermore, when dealing with 2 types of spot urine nutritional component indexes of the magnesium/creatinine ratio and the urea-nitrogen/creatinine ratio, 4 diet evaluation patterns of comments relating to fruits, seaweed, dairy products, and protein may be prepared.

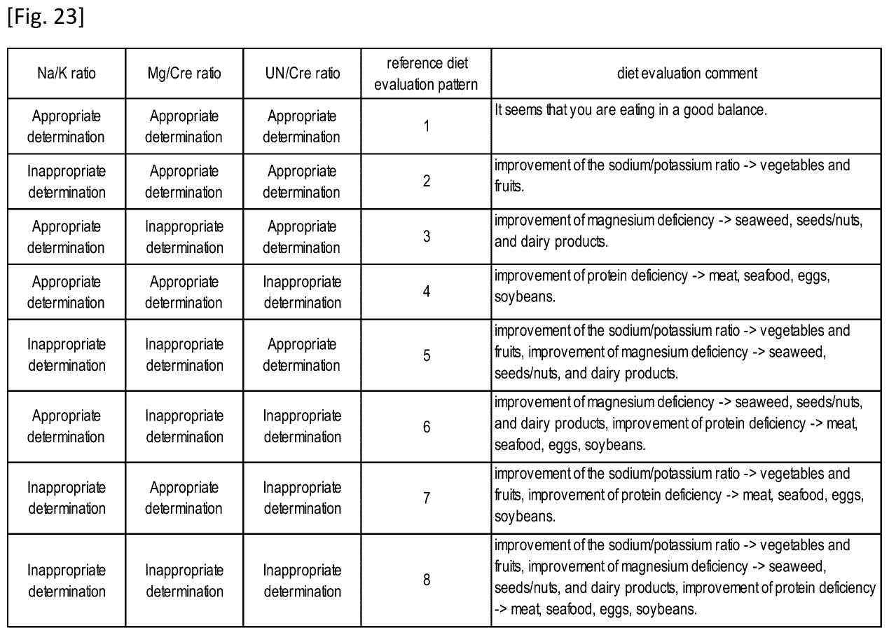

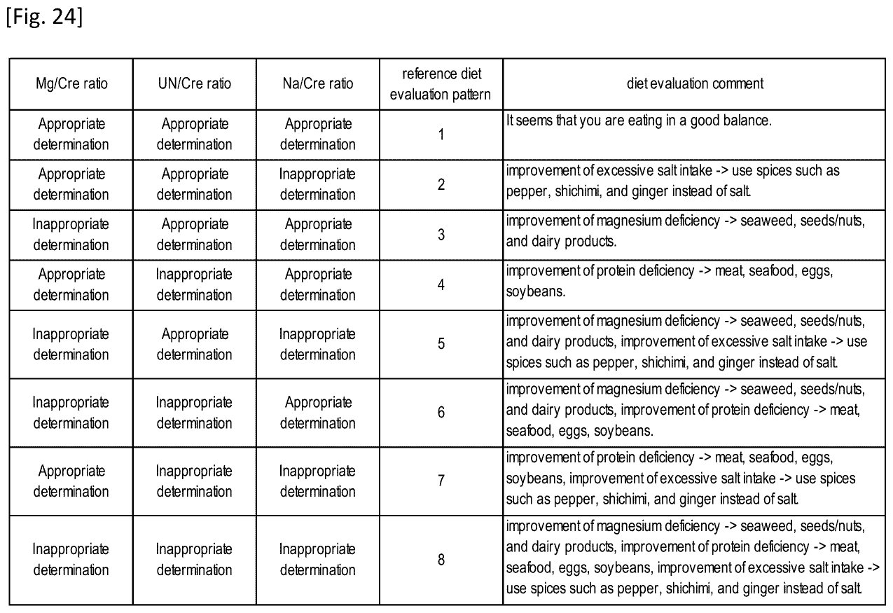

As shown in FIG. 23, when dealing with 3 types of spot urinary nutritional component indexes of the sodium/potassium ratio, the magnesium/creatinine ratio, and the urea-nitrogen/creatinine ratio, 8 diet evaluation patterns of comments related to salt, vegetables and fruits, fruits, seaweed, dairy products, and protein may be prepared. In addition, as shown in FIG. 24, when dealing with 3 types of spot urine nutritional component indexes of the magnesium/creatinine ratio, the urea-nitrogen/creatinine ratio, and the sodium/creatinine ratio, 8 diet evaluation patterns of comments related to fruits, seaweeds, dairy products, protein, and salt may be prepared. The combination of spot urine nutritional component indexes may be appropriately selected from 4 types of the sodium/potassium ratio, the magnesium/creatinine ratio, the urea-nitrogen/creatinine ratio, and the sodium/creatinine ratio shown in FIG. 1. And, other nutritional component indexes may be added to these.

Here, as the number of spot urine nutritional component indexes to be evaluated increases, the nutritional status of the human can be evaluated from various perspectives and as the number of spot urine nutritional component indexes decreases, the human nutritional status can be evaluated from a minimal perspective.

### INDUSTRIAL APPLICABILITY

As described above, the dietary habit evaluation system and the dietary habit evaluation method according to the present invention are useful as a system and method for evaluating the balance of the diet of the human using one spot urine of the human from the nutritional component indexes of one spot urine of the human, and use it to improve the dietary habit of the human and food education. It is effective as the dietary habit evaluation system and the dietary habit evaluation method that can easily and entirely evaluate human dietary habit by using a human spot urine.

### REFERENCE SIGNS LIST

1 DIETARY HABIT EVALUATION SYSTEM
10 EVALUATION TERMINAL DEVICE
11 NETWORK
12 USER TERMINAL DEVICE
101 ACQUISITION CONTROL UNIT
102 DETERMINE CONTROL UNIT
103 DECISION CONTROL UNIT
104 OUTPUT CONTROL UNIT

1. A dietary habit evaluation system comprising:
an acquisition control unit acquiring spot urine nutritional component indexes contained in one spot urine excreted by a human, 4 types of the spot urine nutritional component indexes being a sodium/potassium ratio indicating a ratio of a sodium ion concentration to a potassium ion concentration, a magnesium/creatinine ratio indicating a ratio of a magnesium ion concentration to a creatinine concentration, an urea-nitrogen/creatinine ratio indicating a ratio of a urea-nitrogen concentration to the creatinine concentration, and a sodium/creatinine ratio indicating a ratio of the sodium ion concentration to the creatinine concentration;
a determination control unit comparing the spot urine nutritional component index with a reference value preset for the spot urine nutritional component index for each of spot urine nutritional component indexes, and determining whether or not the spot urine nutritional component index is an appropriate determination or an inappropriate determination for each of the spot urine nutritional component indexes based on the determination result of whether or not the spot urine nutritional component index is the reference value or more;
a decision control unit deciding a reference diet evaluation pattern of a combination of reference determination results matching each of the determination results for each of the determined spot urine nutritional component indexes in 16 types of reference diet evaluation patterns formed by combining the reference determination results of the appropriate determination or the inappropriate determination for each of the spot urine nutritional component indexes, and acquiring a diet evaluation numerical value associated with the reference diet evaluation pattern used for the determination, the diet evaluation numerical value setting to a higher value the more appropriate determinations of the reference determination results of the reference diet evaluation pattern;
an output control unit outputting a diet evaluation comment improving the balance of the diet of the human for the diet evaluation pattern; and
a questionnaire control unit receiving an input of a frequency of a bad habit being a habit of consuming fried foods or the habit of consuming sweets, acquiring a bad habit numerical value corresponding to the input frequency of the bad habit, the bad habit numerical value being set to be lower as the frequency of the good habit is higher, and outputting a total value by adding the bad habit numerical value to the diet evaluation numerical value.

2. (canceled)

3. (canceled)

4. The dietary habit evaluation system according to claim 1, wherein:
a questionnaire of the frequency of the bad habit being the habit of consuming fried foods or the habit of consuming sweets is obtained by getting the human to answer how often he consumes them in a week for each of the habit of consuming fried foods or the habit of consuming sweets.

5. The dietary habit evaluation system according to claim 1, wherein:
the questionnaire control unit receives an input of a frequency of a good habit being a habit of consuming soybeans, or a habit of consuming seafood, acquiring a good habit numerical value corresponding to the input frequency of the good habit, the good habit numerical value being set to be higher as the frequency of the good habit is higher, and outputs a total value by adding the good habit numerical value to the diet evaluation numerical value.

6. The dietary habit evaluation system according to claim 5, wherein:
a questionnaire of the frequency of the good habit being the habit of consuming soybeans or the habit of consuming seafood is obtained by getting the human to answer how often he consumes them in a week for each of the habit of consuming soybeans or the habit of consuming seafood.

7. The dietary habit evaluation system according to claim 1, wherein:
a BMI control unit receiving an input of a BMI of the human, acquiring a BMI numerical value corresponding to the input BMI, the BMI numerical value being set to a higher value as the BMI is closer to normal, and outputting a total value by adding the BMI numerical value to the diet evaluation numerical value and the bad habit numerical value.

8. The dietary habit evaluation system according to claim 5, wherein:
a BMI control unit receiving an input of a BMI of the human, acquiring a BMI numerical value corresponding to the input BMI, the BMI numerical value being set to a higher value as the BMI is closer to normal, and outputting a total value by adding the BMI numerical value to the diet evaluation numerical value, the bad habit numerical value, and the good habit numerical value.

9. (canceled)

10. (canceled)

11. (canceled)

12. (canceled)

13. A dietary habit evaluation method comprising:
an acquisition control step for acquiring spot urine nutritional component indexes contained in one spot urine excreted by a human, 4 types of the spot urine nutritional component indexes being a sodium/potassium ratio indicating a ratio of a sodium ion concentration to a potassium ion concentration, a magnesium/creatinine ratio indicating a ratio of a magnesium ion concentration to a creatinine concentration, an urea-nitrogen/creatinine ratio indicating a ratio of a urea-nitrogen concentration to the creatinine concentration, and a sodium/creatinine ratio indicating a ratio of the sodium ion concentration to the creatinine concentration;
a determination control step for comparing the spot urine nutritional component index with a reference value preset for the spot urine nutritional component index for each of spot urine nutritional component indexes, and determining whether or not the spot urine nutritional component index is an appropriate determination or an inappropriate determination for each of the spot urine nutritional component indexes based on the determination result of whether or not the spot urine nutritional component index is the reference value or more;
a decision control step for deciding a reference diet evaluation pattern of a combination of reference determination results matching each of the determination results for each of the determined spot urine nutritional component indexes in 16 types of reference diet evaluation patterns formed by combining the reference determination results of the appropriate determination or the inappropriate determination for each of the spot urine nutritional component indexes, and acquiring a diet evaluation numerical value associated with the reference diet evaluation pattern used for the determination, the diet evaluation numerical value setting to a higher value the more appropriate determinations of the reference determination results of the reference diet evaluation pattern;
an output control step for outputting a diet evaluation comment improving the balance of the diet of the human for the diet evaluation pattern; and
a questionnaire control step for receiving an input of a frequency of a bad habit being a habit of consuming fried foods or the habit of consuming sweets, acquiring a bad habit numerical value corresponding to the input frequency of the bad habit, the bad habit numerical value being set to be lower as the frequency of the good habit is higher, and outputting a total value by adding the bad habit numerical value to the diet evaluation numerical value.

14. (canceled)

15. (canceled)

16. The dietary habit evaluation system according to claim 4, wherein:
the questionnaire control unit receives an input of a frequency of a good habit being a habit of consuming soybeans, or a habit of consuming seafood, acquiring a good habit numerical value corresponding to the input frequency of the good habit, the good habit numerical value being set to be higher as the frequency of the good habit is higher, and outputs a total value by adding the good habit numerical value to the diet evaluation numerical value.

17. The dietary habit evaluation system according to claim 16, wherein:
a questionnaire of the frequency of the good habit being the habit of consuming soybeans or the habit of consuming seafood is obtained by getting the human to answer how often he consumes them in a week for each of the habit of consuming soybeans or the habit of consuming seafood.

18. The dietary habit evaluation system according to claim 17, comprising:
a BMI control unit receiving an input of a BMI of the human, acquiring a BMI numerical value corresponding to the input BMI, the BMI numerical value being set to a higher value as the BMI is closer to normal, and outputting a total value by adding the BMI numerical value to the diet evaluation numerical value, the bad habit numerical value, and the good habit numerical value.

19. The dietary habit evaluation system according to claim 16, comprising:
a BMI control unit receiving an input of a BMI of the human, acquiring a BMI numerical value corresponding to the input BMI, the BMI numerical value being set to a higher value as the BMI is closer to normal, and outputting a total value by adding the BMI numerical value to the diet evaluation numerical value, the bad habit numerical value, and the good habit numerical value.

20. The dietary habit evaluation system according to claim 6, comprising:
a BMI control unit receiving an input of a BMI of the human, acquiring a BMI numerical value corresponding to the input BMI, the BMI numerical value being set to a higher value as the BMI is closer to normal, and outputting a total value by adding the BMI numerical value to the diet evaluation numerical value, the bad habit numerical value, and the good habit numerical value.

21. The dietary habit evaluation system according to claim 7, wherein:
a questionnaire of the frequency of the bad habit being the habit of consuming fried foods or the habit of consuming sweets is obtained by getting the human to answer how often he consumes them in a week for each of the habit of consuming fried foods or the habit of consuming sweets.

22. The dietary habit evaluation system according to claim 21, wherein:
the questionnaire control unit receives an input of a frequency of a good habit being a habit of consuming soybeans, or a habit of consuming seafood, acquiring a good habit numerical value corresponding to the input frequency of the good habit, the good habit numerical value being set to be higher as the frequency of the good habit is higher, and outputs a total value by adding the good habit numerical value to the diet evaluation numerical value.

23. The dietary habit evaluation system according to claim 22, wherein:
a questionnaire of the frequency of the good habit being the habit of consuming soybeans or the habit of consuming seafood is obtained by getting the human to answer how often he consumes them in a week for each of the habit of consuming soybeans or the habit of consuming seafood.

24. The dietary habit evaluation system according to claim 1, wherein:
the determination control unit determines that the sodium/potassium ratio is appropriate when the sodium/potassium ratio is less than the reference value, and determines that the sodium/potassium ratio is inappropriate when the sodium/potassium ratio is the reference value or more,
the determination control unit determines that the magnesium/creatinine ratio is appropriate when the acquired magnesium/creatinine ratio is the reference value or more, and determines that the magnesium/creatinine ratio is inappropriate when the acquired magnesium/creatinine ratio is less than the reference value,
the determination control unit determines that the urea-nitrogen/creatinine ratio is appropriate when the acquired urea-nitrogen/creatinine ratio is the reference value or more, and determines that urea-nitrogen/creatinine ratio is inappropriate when the acquired the urea-nitrogen/creatinine ratio is less than the reference value,
the determination control unit determines the sodium/creatinine ratio is appropriate when the acquired sodium/creatinine ratio is less than the reference value, and determines that the sodium/creatinine ratio is inappropriate when the sodium/creatinine ratio is the reference value or more.

25. The dietary habit evaluation system according to claim 1, wherein:
it is determined that the lower the sodium/potassium ratio, the less the intake of salt and the higher the intake of vegetables and fruits consumed in the diet of human,
the reference value of the sodium/potassium ratio is determined based on a desired daily intake of sodium and a desired daily intake of potassium of a human,
it is determined that the higher the magnesium/creatinine ratio, the higher the intake of seaweed, seeds, and dairy products consumed in the diet of human,
the reference value of the magnesium/creatinine ratio is determined based on a desired daily intake of magnesium for a human corresponding to the weight of the human,
it is determined that the higher the urea-nitrogen/creatinine ratio, the higher the intake of meat, seafood, eggs, and soybeans consumed in the diet of human,
the reference value of the urea-nitrogen/creatinine ratio is determined based on a desired daily intake of protein for a human corresponding to the weight of the human,
it is determined that the lower the sodium/creatinine ratio, the less the intake of salt consumed in the diet of human, and
the reference value of the sodium/creatinine ratio is determined based on a desired daily sodium intake of a human corresponding to the body weight of the human.

26. The dietary habit evaluation system according to claim 1, wherein:
the diet evaluation comment when the sodium/potassium ratio is the inappropriate determination is an inappropriate determination comment that lists necessary foods of vegetables and fruits to improve the sodium/potassium ratio,
the diet evaluation comment when the magnesium/creatinine ratio is the inappropriate determination is an inappropriate determination comment that lists necessary foods of seaweed, seeds/nuts, and dairy products to improve the magnesium/creatinine ratio,
the diet evaluation comment when the urea-nitrogen/creatinine ratio is the inappropriate determination is an inappropriate determination comment that lists necessary foods of meat, seafood, eggs, and soybeans to improve the urea-nitrogen/creatinine ratio,
the diet evaluation comment when the sodium/creatinine ratio is the inappropriate determination is an inappropriate determination comment that lists necessary foods to improve the sodium/creatinine ratio, and
the output control unit outputs the diet evaluation comment by the inappropriate determination comment corresponding to the spot urine nutritional component index of the inappropriate determination for the reference diet evaluation pattern with the inappropriate determination for each spot urine nutritional component index with the inappropriate determination, and by adding the obtained inappropriate determination comments.

27. The dietary habit evaluation system according to claim 2, wherein:
the diet evaluation comment when the sodium/potassium ratio is the inappropriate determination is an inappropriate determination comment that lists necessary foods of vegetables and fruits to improve the sodium/potassium ratio,
the diet evaluation comment when the magnesium/creatinine ratio is the inappropriate determination is an inappropriate determination comment that lists necessary foods of seaweed, seeds/nuts, and dairy products to improve the magnesium/creatinine ratio,
the diet evaluation comment when the urea-nitrogen/creatinine ratio is the inappropriate determination is an inappropriate determination comment that lists necessary foods of meat, seafood, eggs, and soybeans to improve the urea-nitrogen/creatinine ratio,
the diet evaluation comment when the sodium/creatinine ratio is the inappropriate determination is an inappropriate determination comment that lists necessary foods to improve the sodium/creatinine ratio, and
the output control unit outputs the diet evaluation comment by the inappropriate determination comment corresponding to the spot urine nutritional component index of the inappropriate determination for the reference diet evaluation pattern with the inappropriate determination for each spot urine nutritional component index with the inappropriate determination, and by adding the obtained inappropriate determination comments.

28. The dietary habit evaluation system according to claim 1, wherein:
the decision control unit acquires a diet evaluation numerical value associated with the reference diet evaluation pattern used for the determination, the diet evaluation numerical value setting to a higher value the more appropriate determinations of the reference determination results of the reference diet evaluation pattern, and
the output control unit outputs the diet evaluation comment for the decided diet evaluation pattern of the human, and the acquired diet evaluation numerical value.
